(19) United States
(12) Patent Application Publication
(10) Pub. No.: US 2026/0325208 A1
(43) Pub. Date: Oct. 22, 2026

(54) PLASMA PROBE AND SYSTEM

(71) Applicant: Erbe Elektromedizin GmbH, Tuebingen (DE)

(72) Inventors: Uwe SCHNITZLER, Tuebingen (DE); Daniel SADLER, Gechingen (DE); Tjark BRANDT, Kusterdingen (DE); Nermin SALKIC, Moessingen (DE)

(21) Appl. No.: 19/653,562

(22) Filed: Apr. 21, 2026

(30) Foreign Application Priority Data
Apr. 22, 2025 (EP) 25171754.2
Aug. 28, 2025 (EP) 25198812.7

(51) Int. Cl. A61B 18/18; A61B 18/00

(52) U.S. Cl. CPC A61B 18/18; A61B 2018/00077; A61B 2018/00589; A61B 2218/002

(57) ABSTRACT

The disclosure relates to a plasma probe for generating a plasma for use in medical applications, in particular for treating tissue by coagulation. The probe has a probe body extending from a proximal probe end to a distal probe end and comprises at least one lumen configured for gas conduction. At the distal probe end, at least one electrode and at least one gas outlet opening are provided. The electrode comprises a flow guiding body that protrudes distally from the probe body at least in part and is provided with at least one guiding surface that is skewed with respect to the longitudinal axis (L). The guiding surface is configured such that a lateral flow component is superimposed on the discharging gas stream, thereby enabling targeted shaping and widening of the plasma zone. This improves, in particular, ignition stability and effectiveness in lateral applications. The invention further relates to a system comprising the plasma probe and a supply unit with a high-frequency generator and a gas source for plasma generation.

This application claims priority to European Patent Application No. 25171754.2 filed Apr. 22, 2025, and European Patent Application No. 25198812.7 filed Aug. 28, 2025, the entirety of each of which are incorporated herein.

The present invention relates to a plasma probe for generating a plasma that is usable in particular in medical applications. The plasma can be used, for example, for surface treatment of human tissue, preferably for coagulation of human tissue of a patient. Furthermore, the invention relates to a system consisting of such a plasma probe and an associated supply unit.

In the field of medicine, plasma probes for generating plasma and applying the plasma to a tissue surface are already known from the prior art in different configurations.

EP 1 682 023 B 1 describes a probe for coagulating tissue, which is configured to feed argon gas into a space between an electrode inside the probe and the tissue. In this probe, the electrode protrudes from the probe at a distal end. At the distal end, a guide device for directing and guiding the gas or plasma is additionally arranged, such that at least a portion of the flowing gas or plasma is deflected into the predetermined direction.

JP 2002-301088 A describes a plasma probe in which a fluid conducting lumen is guided to a distal electrode. Within the plasma probe, plasma is generated by the combination of a fluid flow and high frequency current. The plasma then discharges through a nozzle opening at the distal end of the probe.

WO 2017/198869 A1 discloses a plasma system in which two electrodes are wound helically around a dielectric body. In particular, these are flat-band electrodes that bear against a circumferential surface of the dielectric body. Openings are provided in the dielectric body through which the gas for plasma generation can escape into the regions between the electrodes in which the electric field is present.

DE 696 33 182 T2 further discloses a catheter having a flat-band electrode extending in a spiral manner.

DE 11 2022 000 926 T5 describes an electro-surgical device having a counter-electrode and an active electrode with a hydrophobic or superhydrophobic surface. This is designed to retain a portion of the gas from a vapor pocket when the active electrode is in a conductive liquid solution and has transitioned into an inactive state.

DE 100 30 111 A1 discloses a probe for electro-surgical treatment of tissue comprising a tube- or hose-shaped feed line for transporting a noble gas from a surface to the distal end, as well as an electrode arranged with a discharge section and an electrical feed line for supplying HF current.

In certain medical applications, such as plasma coagulation of difficult to access and convoluted areas, for example the stomach, positioning and alignment of the plasma probe relative to the patient's tissue continues to be challenging.

In particular, in monopolar operation of the plasma probe—meaning that the plasma probe has an active electrode and a counter-electrode is attached remotely on the patient's body—a precise distance between the probe and the tissue to be treated must be maintained. Only under these conditions can the high-frequency currents (HF currents) flow or ignite between the active electrode and the tissue within the gas stream or ignite, respectively, and thereby generate plasma. A distance that is too large makes ignition or flow, respectively, of the HF currents between the active electrode and the tissue difficult, with the result that no plasma is generated and no therapeutic effect is achieved. A distance that is too small, on the other hand, increases the risk of unintended tissue contact with the active electrode, which can lead to unintended injury of the tissue. In most known plasma probes, the main gas stream occurs in the axial direction, which makes lateral treatment difficult, because in this region comparatively less plasma tends to be generated.

Starting from this, the object of the present invention is to provide an improved plasma probe, with which larger tissue areas can be treated with plasma. In particular, improved ignition behavior of the plasma probe at different distances as well as at different angles between the plasma probe and the tissue is to be achieved.

This object is achieved by plasma probes disclosed herein.

An example plasma probe according to the disclosure has a probe body that extends from a proximal probe end toward a distal probe end. The probe body is preferably configured as a flexible hose or as a flexible tube.

At least one lumen is formed in the probe body, which is configured for conducting a gas such as argon. At the distal probe end of the plasma probe, at least one electrode is arranged. In addition, at least one gas outlet opening is formed at the distal probe end. At least one lumen opens into this gas outlet opening such that a gas stream can discharge from the probe.

Within the context of the invention, plasma is understood as an electrically conductive ionized gas that is generated in particular at atmospheric pressure by applying a high frequency field. Preferably, the gas used is argon such that the plasma probe generates argon plasma.

In particular, the at least one electrode has a flow guiding body that extends along a longitudinal axis and protrudes distally from the probe body, at least in part. The flow guiding body preferably consists of an electrically conductive material. The flow guiding body may be made, for example, of metal such as stainless steel, tungsten, aluminum, copper, silver, and the like, or of carbon fibers or other carbon elements such as graphite, graphene, and the like. The flow guiding body may also be provided with a thermally and/or electrically conductive coating such as silver. At least in part, the flow guiding body can form the at least one electrode. The electrode configured as a flow guiding body can be used both for shaping the gas or plasma stream, respectively, and for generating the plasma itself.

The flow guiding body has at least one guiding surface that extends along an extension direction. The guiding surface is configured such that it specifically deflects and shapes the gas stream. Preferably, the guiding surface extends along an extension direction that is skewed with respect to the longitudinal axis. In a particularly preferred embodiment, the extension direction is skewed at every point. The extension direction is defined as the direction in which the (radially outermost) edge of the guiding surface continues in a side projection, the side projection being taken in a radial direction with respect to the longitudinal axis. Preferably, the extension direction of the guiding surface is skewed with respect to the longitudinal axis at every point of the guiding surface, i.e. the extension direction does not intersect the longitudinal axis. The guiding surface extends in particular in a spiral manner around the longitudinal axis of the flow guiding body. The radial distance between the guiding surface and the longitudinal axis can remain at least substantially constant in the distal direction or can continuously decrease or increase. The extension direction of the guiding surface is in particular (at every location) at an inclination angle relative to the longitudinal axis of the flow guiding body. This geometry enables targeted deflection and shaping of the existing gas stream. In particular, the guiding surface extends from a section of the flow guiding body located inside the probe body to a section of the flow guiding body located outside the probe body.

Before the gas stream impinges on the guiding surface or guiding surfaces of the flow guiding body, the gas stream flows at least substantially axially, i.e., parallel to the longitudinal axis from proximal to distal. Configuring the electrode as a flow guiding body makes it possible to superimpose a lateral component on the gas stream in addition to the axial component and to deflect it outwards and thereby expand it. As a result, a spray cone is formed, the diameter of which can be varied as a function of the (axial) flow velocity of the gas stream or plasma flow. At lower flow velocity, the diameter of the spray cone is larger than at higher flow velocity. If a larger treatment area is desired, the diameter of the existing gas cone (spray cone) can be increased by reducing the flow velocity of the gas.

In a preferred embodiment, the flow guiding body has at least one guiding surface that extends in a helical or screw-like manner and winds around the longitudinal axis of the flow guiding body. The external shape of the flow guiding body thereby resembles a thread or the cutting edge of a drill. By this configuration, the discharging gas stream is specifically deflected outwardly and expanded so that a spray cone is formed whose diameter can be varied depending on the flow velocity.

A particular feature of the present invention is the provision of at least one guiding surface by means of which, in addition to an axial flow component, a lateral flow component is superimposed on the discharging gas stream. This may be achieved, in particular, by a skewed arrangement of the guiding surface. Owing to the skewed arrangement, the gas stream expands laterally, thereby producing a broader plasma formation.

This is advantageous, in particular, for treatment of larger tissue areas or for lateral application. In addition, the gas stream flows past the (radially outer) edge of the guiding surface, where the high frequency field strength is concentrated. As a result, improved lateral plasma generation can be achieved at the (outer) edges of the guiding surfaces. In this way, the lateral ignition stability of the plasma can be improved.

The flow guiding body preferably has at least one (thread-like) flow groove. In particular, the (thread-like) flow groove has, at least in part, a constant, increasing and/or decreasing winding radius relative to the longitudinal axis in the distal direction. Preferably, the winding radius of the flow groove decreases from the proximal end toward the distal end of the flow guiding body. The guiding surface(s) is/are preferably formed by the flank of the respective flow groove on which the (axial) gas stream impinges, i.e., by the flank facing the gas stream. Together with the probe body, i.e., the jacket of the hose or tube, the flow groove preferably forms the respective gas outlet opening.

The edges of the flow groove, in particular the transition between the flank of the flow groove and the remaining outer surface of the flow guiding body, are preferably configured as sharp edges or acute-angled edges. These sharp-edged and/or acute-angled edge regions cause a local increase in the electrical HF field strength, whereby ignition of the plasma can be initiated selectively at these locations.

After ignition, guidance and maintenance of the plasma discharge take place along the respective guiding surface, i.e., along the flank of the flow groove facing the gas stream. Since the flow guiding body is simultaneously configured as an electrode, the plasma flowing along the flank is continuously supplied with high-frequency current, whereby the plasma discharge can be stabilized and extended.

In a preferred embodiment, two, three or more gas outlet openings are formed at the distal probe end of the plasma probe. Accordingly, in this embodiment, the flow guiding body has two, three or more guiding surfaces, each assigned to a respective gas outlet opening and arranged such that the gas stream impinges on the respective guiding surface. As a result, a more uniform gas distribution can be achieved.

With multiple gas outlet openings, the flow guiding body correspondingly has multiple flow grooves, e.g., two, three or more, which can be formed in the outer surface of the flow guiding body. These flow grooves preferably extend helically or in a screw-like manner around the longitudinal axis of the flow guiding body. In such cases, the flow grooves can form multiple gas outlet openings together with the jacket of the probe body.

The flow guiding body has a first section arranged inside the probe body and a second section arranged outside the probe body. The guiding surface(s) or the flow groove(s) can extend along a subsection, preferably along most or all of the outer surface of the flow guiding body. They can extend in the first section, in the second section, or in both sections.

The cross-sectional shape of the flow grooves may be asymmetric or symmetric, for example slot-shaped, elliptical, circular, or kidney-shaped. The cross-sectional plane extends transversely, i.e., at least substantially orthogonally to the extension direction of the guiding surface.

The probe body is preferably configured as a flexible hose. During manufacture of the plasma probe, the flow guiding body can be introduced into the hose, the gas outlet openings being formed by the wall of the probe body and the flow groove. This enables simple and cost-effective manufacture of the distal structure of the plasma probe.

The guiding surfaces preferably extend spirally around the longitudinal axis of the flow guiding body. Where multiple guiding surfaces are provided, they are preferably uniformly distributed around the circumferential surface and extend helically. This results in uniform lateral widening of the gas jet upon discharging.

The geometric shape of the flow guiding body can be substantially cylindrical, frustoconical, or semi-ellipsoidal. This enables application-specific optimization of the flow and plasma parameters.

The flow guiding body preferably forms the electrode entirely, at least in certain regions, such that the electrode can serve both to widen the gas stream and to provide high-frequency currents in the gas stream for generating the plasma.

In a preferred embodiment, the plasma probe is additionally configured to dispense a jet-forming fluid. For this purpose, the plasma probe can have a jet-forming device for dispensing a jet-forming fluid, comprising at least one jet-forming opening through which the jet-forming fluid can be dispensed at the distal probe end. The jet-forming device may be a water jet-forming device. This makes it possible, before and/or after plasma treatment, to inject tissue of the patient (submucosal injection) using the plasma probe, i.e., to introduce jet-forming fluid selectively into or under a tissue layer. As a result, the tissue to be treated can be locally raised or separated from deeper layers. The injection serves, in particular, to protect sensitive structures during an intervention, to improve visibility and accessibility of the surgical field, or to support targeted application of energy, such as plasma or high-frequency treatment. The jet-forming fluid can preferably be dispensed at the distal probe end in the form of a jet. The jet-forming fluid may be dispensed at a pressure of up to 80 bar, preferably at a pressure between 20 bar and 60 bar. The resulting jet may comprise a velocity in the range from 35 m/s to 72 m/s.

In one embodiment, the jet-forming device can comprise a central channel formed in the flow guiding body and configured to conduct a jet-forming fluid. The channel preferably extends along the longitudinal axis of the flow guiding body and opens into one or more jet-forming openings at a nozzle. For this purpose, the channel is connected to a separate lumen of the probe so that the jet-forming fluid can be conveyed from the proximal probe end to the distal probe end independently of the gas stream.

In a preferred embodiment, the jet-forming device has a nozzle element that is arranged at least partially in a through channel of the flow guiding body. The through channel extends from a distal end region of the flow guiding body to a proximal end region of the flow guiding body along the longitudinal axis. The outer diameter of the nozzle element can be smaller than the inner diameter of the through channel, or can at least substantially correspond to the inner diameter of the through channel. In particular, the nozzle element can be cylindrical, preferably hollow-cylindrical.

A nozzle channel can be provided in the nozzle element, extending along the longitudinal axis from the proximal end to the distal end of the nozzle element. One or more jet-forming openings are arranged at the distal end of the nozzle element. The nozzle channel preferably narrows toward the jet-forming opening.

The nozzle element is preferably fluidically connected at its proximal end to a capillary tube so that the nozzle element can be supplied with jet-forming fluid via the capillary tube. The capillary tube preferably extends from the proximal probe end to the distal probe end. The capillary tube is made, for example, of stainless steel.

Using a dedicated nozzle element can significantly reduce assembly effort and simplify manufacture of the plasma probe. The nozzle element can first be connected to the capillary tube. For this purpose, for example, the capillary tube can first be widened at the distal end such that the nozzle element can be inserted into the capillary tube from the distal side. The nozzle element preferably sits in the capillary tube without play. Alternatively, the nozzle element can have, at its proximal end, a widened opening in which the distal end of the capillary tube can be received, such that the nozzle element can be fitted onto the capillary tube at the distal end without play. In order to achieve a fluid-tight connection between the nozzle element and the capillary tube, the two are connected to one another by a suitable joining technique, for example by producing a circumferential ring weld by laser welding or by a comparable method.

After the nozzle element has been fluidically connected to the capillary tube, the flow guiding body can be fitted onto the distal end of the nozzle element such that the nozzle element and, for example, also a subsection of the capillary tube are arranged in the through channel of the flow guiding body. The flow guiding body can then be fastened in its desired fixing position on the capillary tube, for example by one or more spot welds.

In a preferred embodiment, the nozzle element can be arranged at least partially in the through channel. The capillary tube can also be arranged partially in the through channel of the flow guiding body such that the connection point between the nozzle element and the capillary tube is arranged within the through channel. The jet-forming fluid can be, for example, a NaCl solution, Ringer's solution, or distilled water.

In a preferred embodiment, the nozzle element is simultaneously configured as an electrode. It consists of an electrically conductive material, in particular a metal such as tungsten or stainless steel. Preferably, the nozzle element is formed in one piece. In this embodiment, the capillary tube can likewise be made of an electrically conductive material, in particular a metal such as stainless steel. Preferably, the flow guiding body is in electrical contact with the nozzle element in this embodiment, so that the flow guiding body can be supplied with high-frequency voltage via the nozzle element and the capillary tube. In this way, the nozzle element can assume a combined function both for fluid dispensing and as an electrode. It is preferred that the nozzle element has higher heat resistance than the capillary tube and/or the flow guiding body. Preferably, the nozzle element consists of a material such as tungsten that is more temperature resistant than the material of the capillary tube and/or the flow guiding body. Since the nozzle element is configured as a separate component, it can be provided with higher heat resistance. Thereby, the nozzle element remains dimensionally stable even over long treatment times. Thus, in particular, the nozzle opening can retain its shape even during long treatments. Alternatively, or additionally, the nozzle element can also be provided with a coating by which the heat resistance of the nozzle element is increased. In this embodiment, the coating consists, for example, of a material having higher heat resistance than the material of the nozzle element. For example, the coating can consist of tungsten.

At the distal end of the flow guiding body, the nozzle element can in particular be arranged such that it protrudes distally from the through channel, which can improve injection, for example with regard to achievable precision and handling.

The plasma probe can further have at least one fixing element, which is arranged proximally of the gas outlet opening inside the probe hose. The fixing element is preferably configured to hold a capillary tube centered in the probe hose and at the same time to ensure an axial fixing between the capillary tube and the probe hose. For this purpose, the fixing element can comprise, for example, one or more support plates that extend radially between the capillary tube and the inner wall of the probe hose. The support plates can be welded or crimped to the capillary tube. On the outer sides of the support plates projections can be provided, which support against the inner wall of the probe hose and thereby reinforce the connection between the capillary tube and probe hose, so that axial displacement of the capillary tube and of the nozzle element and/or of the flow guiding body relative to the probe hose is prevented. By this configuration it is achieved that the probe hose is held centered and axially fixed on the capillary tube, which ensures a reliable and long-term stable arrangement.

The fixing element can be configured as a separate component. Additionally, or alternatively, the fixing element can also be provided directly on the flow guiding body, for example by geometric features such as elevations or barbed (fir-tree-shaped) structures on the outer circumference. These features are preferably arranged in a section of the flow guiding body that in the assembled state is enclosed in the circumferential direction by the probe hose. The fixing element can be prevent that the components forming the electrode—namely the nozzle element, the capillary tube and the flow guiding body—are pulled out of the probe hose during use.

The surface of the flow guiding body preferably has depressions. Preferably, the depressions are configured as a plurality of grooves or notches. The depth of the depressions is preferably (substantially) smaller than the depth of the flow grooves. The depressions cause the surface of the electrode or flow guiding body to have additional edges, which lead to local field enhancement and thus improve the ignition behavior of the plasma. The depressions can be formed over the entire surface or selectively, in particular in the region outside the probe body. Preferably, however, the depressions are formed exclusively on the surface of the flow guiding body and not in the flow grooves.

The extension direction of the guiding surface or guiding surfaces is/are arranged at a predetermined inclination angle relative to the longitudinal axis. The inclination angle is in particular between 90° and 0°, preferably between 65° and 15°, particularly preferably between 65° and 35°.

Furthermore, in a longitudinal section, the guiding surface can have, at least in a central region of the guiding surface, a tangent arranged at a predetermined angle of attack relative to the radial direction of the flow guiding body. The longitudinal section is a section along the longitudinal axis through the flow guiding body. The guiding surface can be at least partially planar or at least partially curved. In longitudinal section, the guiding surface can be linear or at least partially curved, for example convexly curved. The angle of attack can in particular be between 0° and 90°, preferably between 30° and 70°. Such an arrangement can selectively influence the direction of gas deflection and improve lateral plasma distribution. The gas forced laterally out of the flow groove can thereby flow past the edge of the flow groove, where the electrical HF field strength is increased, thereby improving the ignition behavior of the plasma.

The object of the invention is further achieved by disclosed systems comprising a supply unit and a plasma probe of the type described above. The previously described features and advantages are likewise applicable to the system according to the invention.

The supply unit comprises an HF generator for supplying the electrode with HF currents and a gas source for providing a plasma gas, preferably an inert gas such as argon. The flow velocity of the gas is preferably adjustable. Within the context of the present application, the term “HF current” designates an alternating current having a frequency in the range of about 4 kHz to 500 kHz, preferably between 300 kHz and 500 kHz. The gas is preferably an inert gas such as argon. The flow velocity at which the gas source provides the gas stream is preferably variable.

The plasma probe is connected to the supply unit such that at least one lumen is fluidically connected to the gas source and a gas stream is discharged from the at least one gas outlet opening. If the plasma probe is further configured to dispense jet-forming fluid at the distal probe end, the supply unit can additionally be configured to supply the plasma probe with jet-forming fluid. The jet-forming fluid is conveyed from the proximal probe end through the capillary tube to the jet-forming opening at the distal probe end. The electrode is electrically connected to the HF generator and can thus be supplied with HF currents.

The plasma probe is configured in particular for monopolar operation. In this case, the HF generator is configured for monopolar operation, wherein the electrode of the plasma probe forms the active electrode and a counter electrode to be attached to the patient's body is provided. The active electrode and the counter electrode are electrically connected to the HF generator.

### BRIEF DESCRIPTION OF THE FIGURES

FIG. 1 shows an embodiment of the system according to the invention;

FIGS. 2 to 6 show embodiments of the distal probe end of the plasma probe in a partially sectional side view including embodiments with cylindrical, frustoconical, and semi-ellipsoidal flow guiding bodies partially arranged inside the probe body, and an embodiment with a cylindrical flow guiding body arranged entirely outside the probe body;

FIGS. 7 to 9 show different configurations of the flow groove and corresponding detailed views including U-shaped, rectangular and V-shaped cross-sectional shapes;

FIG. 10 shows a cross-section through the probe body;

FIGS. 11, 12 show exemplary configurations of the distal probe end of the plasma probe in perspective view;

FIG. 13 shows a flow profile at high flow velocity;

FIG. 14 shows a flow profile at low flow velocity;

FIG. 15 shows an example of assembly of the flow guiding body at the distal probe end;

FIG. 16 shows an embodiment of the distal probe end of the plasma probe in longitudinal section;

FIG. 17 shows a detailed view of the fixing element in longitudinal section;

FIG. 18 shows a detailed view of the fixing element in cross-section.

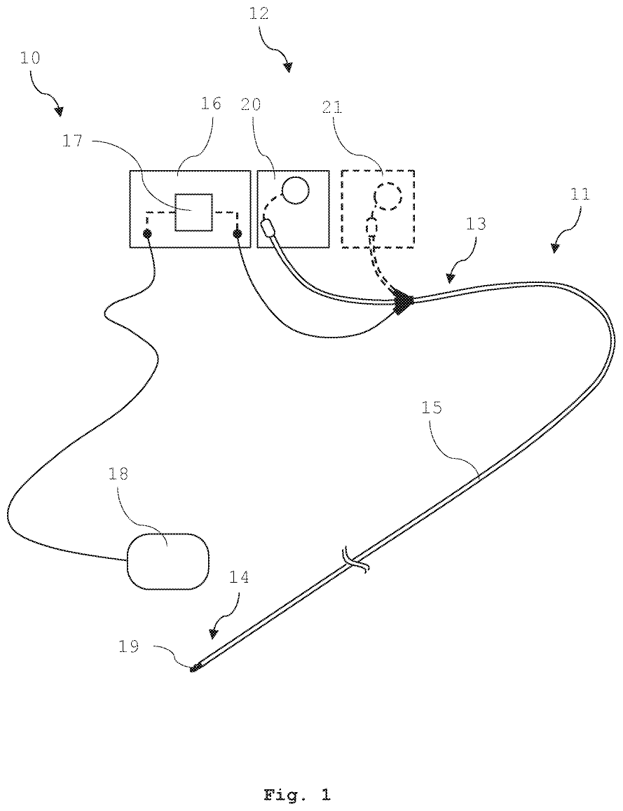

FIG. 1 shows an example of the system 10 according to the invention comprising a plasma probe 11 and a supply unit 12. The plasma probe 11 extends from a proximal probe end 13 to a distal probe end 14. It has a probe body 15 formed from a flexible tube or hose.

At the proximal probe end 13, the plasma probe 11 has a plurality of connectors by which it can be connected to the supply unit 12.

The supply unit 12 has an HF unit 16 with an HF current generator 17. The HF unit 16 has a connector for electrically connecting a counter electrode 18 that can be attached to a body part of the patient. In addition, a connector for the plasma probe 11 is provided, via which a feed line 19 to the active electrode can be connected. The feed line 19 conducts the HF current from the proximal probe end 13 to the distal probe end 14 where the electrode is supplied with the HF current. The system 10 shown is configured for monopolar operation in which the electrode of the plasma probe 11 functions as an active electrode and the circuit is closed via the counter electrode 18.

Furthermore, the supply unit 12 comprises a gas source 20 for providing a plasma generation gas, for example argon. The gas source 20 is preferably configured such that the discharging flow velocity is adjustable. The plasma probe 11 is fluidically connected to the gas source 20 via the proximal probe end 13.

Optionally, the plasma probe 11 can also be configured to output a jet-forming fluid at the distal probe end 14. For this purpose, the supply unit 12 can have a jet-forming fluid source 21. The jet-forming fluid source 21 is shown in dashed lines in FIG. 1 because it is purely optional. The jet-forming fluid source 21 can provide a jet-forming fluid, which can be, for example, a sodium chloride solution, distilled water, or a Ringer's solution.

The HF unit 16, the gas source 20 and the jet-forming fluid source 21 can be configured as separate units or as a combined unit.

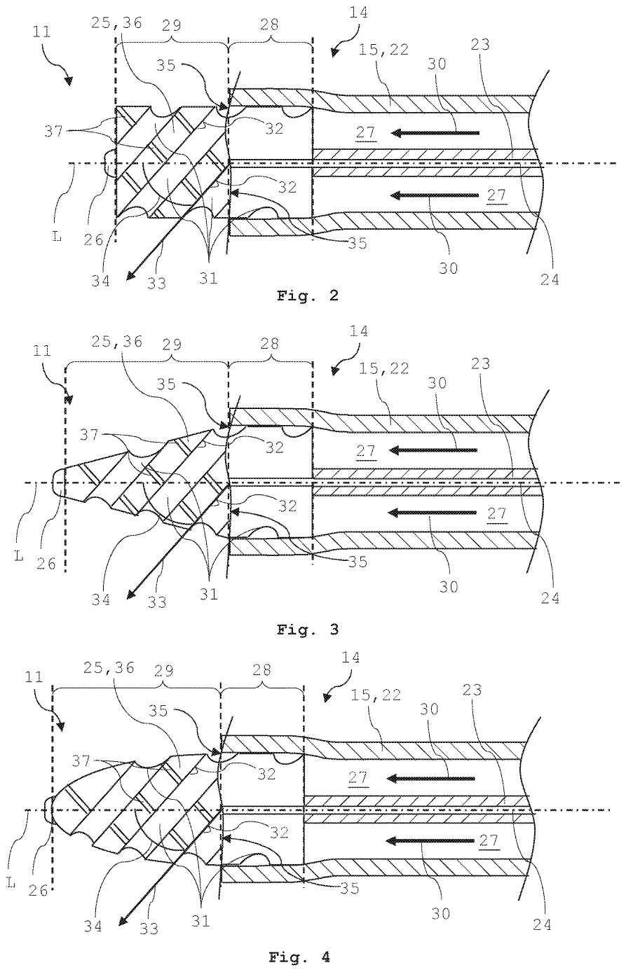

FIG. 2 shows an example of a configuration of the distal probe end 14 of the plasma probe 11 in a partially sectional side view. In the illustrated examples, the probe body 15 of the plasma probe 11 is configured as a hose or flexible tube having a wall 22. The wall 22 of the plasma probe 11 can be made of a flexible plastic material. Likewise, the wall 22 can have a reinforcement structure, which, however, is not shown in FIGS. 2 to 6. In the examples shown in FIGS. 2 to 6, the electrical feed line 23 is shown arranged centrally relative to the wall 22.

In the embodiment shown in FIGS. 2 to 6, the electrical feed line 23 comprises a channel 24, which extends from the proximal probe end 13 to the distal probe end 14 and opens at the distal probe end 14 into a flow guiding body 25. The flow guiding body 25 continues the channel 24 as far as a distally arranged jet-forming device 26 from which the jet-forming fluid can discharge.

Around the electrical feed line 23, a lumen 27 is formed through which the gas stream 30 is guided from the proximal to the distal probe end 13 and 14. Unlike what is shown in FIGS. 2 to 6, a plurality of lumens can also be formed around the electrical feed line 23 through which, for example, multiple gas streams can be transported from the proximal to the distal probe end.

In this example, the flow guiding body 25 is cylindrical. It has a first section 28 located inside the probe body 15 and a second section 29 protruding distally from the probe body 15. The flow guiding body 25 extends along a longitudinal axis L. On the surface of the flow guiding body 25, flow grooves 31 are present, which deflect the gas stream 30 impinging on the flow guiding body 25. The flow grooves 31 have guiding surfaces 32 that extend along an inclined extension direction 33. The inclination relative to the longitudinal axis L is defined by an inclination angle 34.

The flow grooves 31 form together with the wall 22 of the probe body 15 gas outlet openings 35 through which the gas stream 30 exits. The outlet direction follows the extension direction 33 of the respective guiding surface 32. As a result, the gas stream can spread laterally depending on its velocity.

The flow guiding body 25 forms the electrode 36. This is supplied with HF current via the electrical feed line 23. The surface of the flow guiding body 25 can additionally have a plurality of depressions 37. For example, in the form of transverse grooves that extend transversely to the extension direction 33 of the flow grooves 31. The depressions 37 generate additional edges that can lead to local field enhancement and thus to improved ignition behavior. The depressions 37 are preferably formed only in the section 29 lying outside the probe body 15 (exposed section).

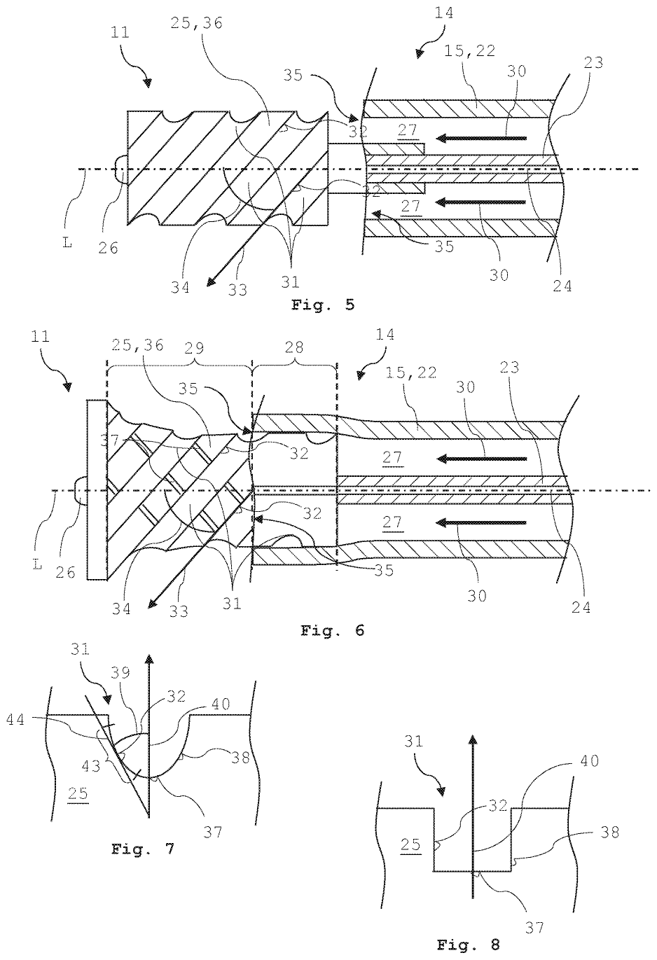

In FIGS. 3 to 6, further embodiments of the flow guiding body 25 are shown. In FIG. 3, the flow guiding body 25 is frustoconical and in FIG. 4 it is semi-ellipsoidal. In the examples shown in FIGS. 3 and 4, the winding radius of the flow grooves 31 decreases from the axial height at which the gas outlet openings are arranged towards the distal end of the flow guiding body 25. In FIG. 5, the flow guiding body, as in FIG. 2, is cylindrical but arranged entirely outside the probe body 15. Around the mouth region in which the gas outlet opening 35 is formed, a sleeve can be provided as schematically shown in FIG. 5, which electrically insulates the electrical feed line from the gas stream. The sleeve consists in particular of an electrically non-conductive material, such as ceramic. FIG. 6 shows the flow guiding body 25 as a frustoconical body whose diameter increases in the distal direction.

FIGS. 7 to 9 show different cross-sections of the flow grooves 31 through the flow guiding body 25 in a detailed view. FIG. 7 shows a U-shaped groove with a bottom section at the crest of the flow groove 31 and an opposite flank 38. The guiding surface 32 is concavely curved in this example. In a central region 43, which in particular comprises neither the edge nor the crest of the flow groove 31, the guiding surface 32 has a tangent 44, which is arranged at an angle of attack 39 relative to the radial direction 40. FIG. 8 shows a rectangular groove form. The guiding surface 32 is planar in this example. In the longitudinal section shown, the guiding surface 32 extends parallel to the radial direction 40. The angle of attack is therefore at least substantially zero in this example. FIG. 9 shows a V-shaped groove form in which no bottom section is present. The guiding surface 32 is also at least substantially planar in this example.

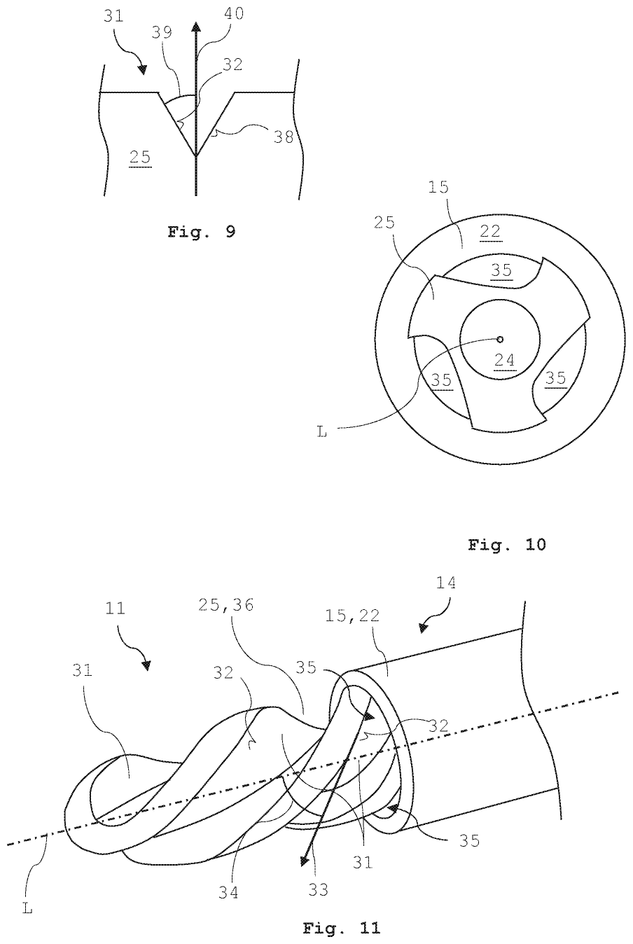

FIG. 10 shows a cross-section through the distal probe end 14. The wall 22 of the plasma probe 11 is in particular a hose wall with a (circular) round or elliptical cross-section. The gas outlet openings 35 are asymmetrical and arranged at a distance from the longitudinal axis L. They are formed by the flow guiding body 25 and the wall 22 of the probe body 15. The flow guiding body 25 is frictionally held (clamped) against the wall 22 and has a larger diameter than the inner diameter of the wall 22. In the flow guiding body 25 a fluid channel 24 is also formed, which extends along the longitudinal axis L from the proximal end of the flow guiding body 25 to the distal end of the flow guiding body 25 and can open there into a jet-forming device 26.

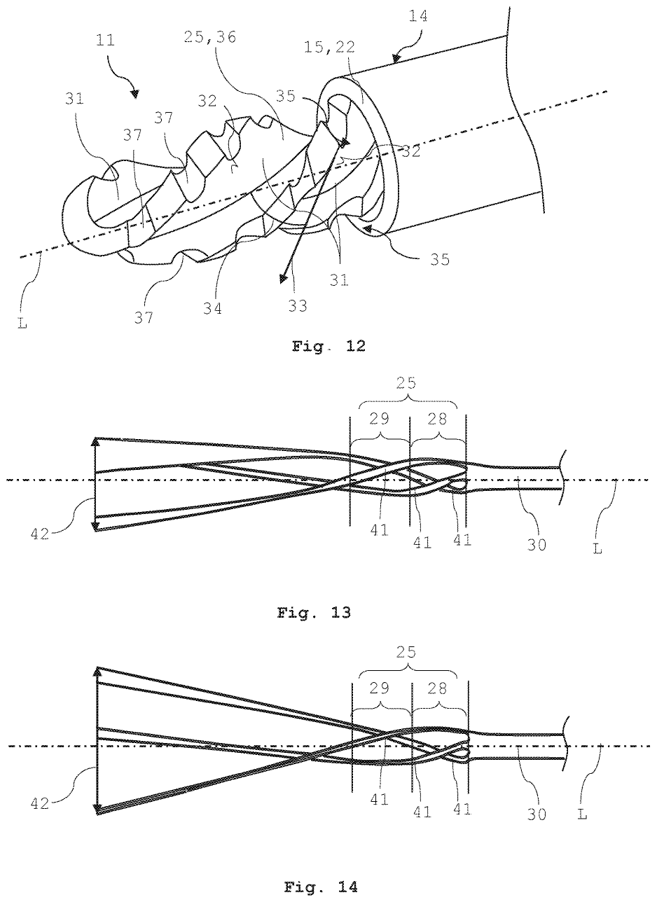

FIGS. 11 and 12 show perspective views of the flow guiding body 25. In FIG. 11 spiral guiding surfaces 32 and multiple depressions 37 on the surface are shown. In FIG. 12 the depressions 37 are formed by transverse grooves. In addition, channels 24 can also be formed in the flow guiding bodies 25 shown in FIGS. 11 and 12 which open at the distal end of the flow guiding bodies into the jet-forming device 26.

FIG. 13 shows the flow profile at high flow velocity of the gas stream 30. The gas stream 30 impinges on the first section 28 of the flow guiding body 25 and is divided into a plurality of partial gas streams 41 that are guided along the winding of the flow grooves 31. The flow is discharged from the probe via the gas outlet openings 35. The diameter 42 of the spray cone is comparatively small.

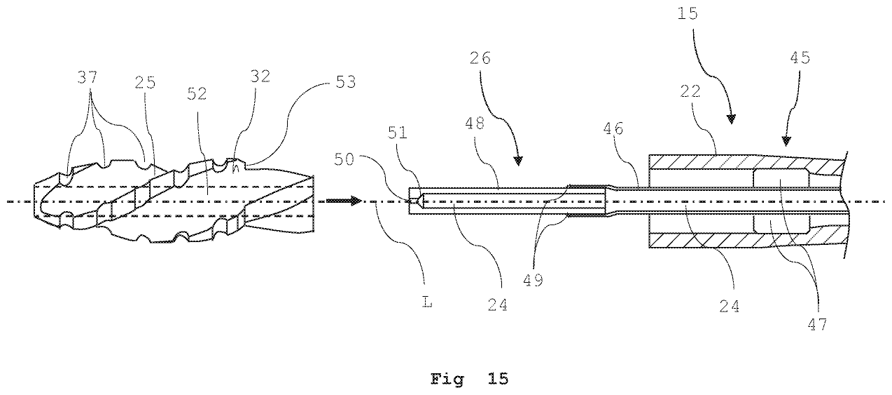

FIG. 14 shows the flow conditions at low flow velocity of the gas stream 30. The spray cone diameter 42 is larger here than in FIG. 13.

FIG. 15 shows an example of assembly of the flow guiding body 25 at the distal probe end 14 of the plasma probe 11. In the example shown in FIG. 15, a fixing element 45 is first attached to the capillary tube 46. The fixing element 45 is in particular fixed in place on the capillary tube 46. For example, the fixing element 45 can be welded or pressed (crimped) onto the capillary tube 46. The fixing element 45 can comprise, for example, one or more support plates 47. The support plates 47 can be rectangular or diamond-shaped or have a comparable shape. In the example shown in FIG. 15, the fixing element 45 has, by way of example, two support plates 47 that extend radially relative to the capillary tube 46. By means of the fixing element 45, the wall 22 of the probe hose 15 is axially fixed to the capillary tube 46. In addition, the capillary tube 46 is thereby centered in the probe hose 15.

Unlike what is shown in FIG. 15, the fixing element 45 can also be configured as a separate component. As described above, the fixing element 45 can additionally or alternatively be provided directly on the flow-guiding body 25, for example, by geometric features such as elevations or barbed (fir-tree-like) structures on the outer circumference. These geometric features are arranged in a section of the flow-guiding body that, in the assembled state, is enclosed in the circumferential direction by the probe hose.

At the distal probe end 14, the capillary tube 46 protrudes from the probe hose 15. In the example shown in FIG. 15, the capillary tube 46 has already been widened in a distal section such that a nozzle element 48 is inserted into the capillary tube 46 from the distal side. The nozzle element 48 sits without play in the widened section of the capillary tube 46. In addition, the nozzle element 48 is fastened to the capillary tube 46 by a circumferential connection 49 so that the jet-forming lumen formed by the capillary tube 46 is sealed and fluidically connected to the nozzle element 48. In contrast, only point connections, such as spot welds, are required between the capillary tube and the flow guiding body.

In the nozzle element 48, the channel 24 formed in the capillary tube 46 is continued through. At the distal end of the nozzle element 48, a jet-forming opening 50 is also arranged through which the jet-forming fluid can be discharged. The nozzle element 48 also has a tapering section 51 that opens into the jet-forming opening 50 of the nozzle element 48.

In FIG. 15, the flow-guiding body 25 is shown, which is pushed onto the nozzle element 48 from the distal end. For this purpose, the flow-guiding body 25 has a through channel 52 shown in dashed lines in FIG. 15, which extends from a proximal end of the flow-guiding body 25 to a distal end of the flow-guiding body 25. The flow-guiding body 25 can, unlike in FIG. 15, have another shape, such as shown in FIGS. 2, 3, 4, 5 and 6, as long as it has a through channel 52.

In the proximal end region of the flow-guiding body 25, a radially extending stop surface 53 is formed on the outside of the flow-guiding body 25, which forms a fixed axial stop for the probe hose 15. In the fully assembled state, the end of the probe hose rests against the stop surface 53. The stop surface 53 defines how far the flow-guiding body 25 can be inserted into the distal probe end 14 of the probe hose 15.

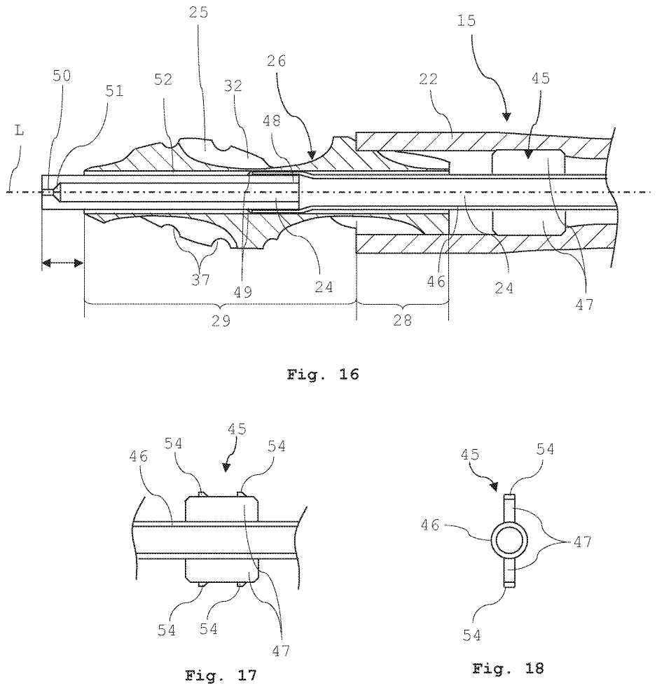

FIG. 16 shows a longitudinal section through the example of the plasma probe shown in FIG. 15 in the assembled state. The stop surface 53 defines the axial length of the first section 28, which in the assembled state is located inside the probe hose 15. The statements made above with respect to the reference signs apply correspondingly to the longitudinal section shown in FIG. 16.

In the assembled state, the nozzle element 48 protrudes distally beyond the flow-guiding body 25 so that the jet-forming opening 50 is arranged spaced apart from the flow-guiding body 25.

FIG. 17 shows a detailed view of the fixing element 45. The fixing element 45 has two radially projecting support plates 47 that are fixed in place on the capillary tube 46. At the outer edges of the support plates 47, these additionally have a plurality of projections 54, which are configured to improve the mechanical connections between the support plates 47 and the wall 22 of the plasma hose 15.

FIG. 18 shows a cross-section of the fixing element 45. Unlike the example shown in FIG. 18, the fixing element 45 can also have 3, 4, 5 or more support plates 47, each of which can extend radially outward from the capillary tube 46. The support plates 47 can be provided at their outer edge with projections 54 or can also be configured smooth. The fixing element 45, shown here by way of example, can also, unlike shown, be configured as a separate component, for example as a sleeve that sits on the outer circumference of the capillary tube and is connected thereto. The sleeve can have at least one support plate extending radially outward. Additionally or alternatively, the fixing element 45 may also be provided directly on the flow-guiding body as described above, for example by geometric features such as projections 54 or barbed (fir-tree-like) structures shown on the outer circumference of the flow-guiding body 25.

The invention relates to a plasma probe 11 for generating a plasma for use in medical applications, in particular for treating tissue by coagulation. The probe 11 has a probe body 15 extending from a proximal probe end 13 to a distal probe end 14 and comprises at least one lumen 27 configured for gas conduction. At the distal probe end 14, at least one electrode 36 and at least one gas outlet opening 35 are provided. The electrode 36 comprises a flow guiding body 25 that protrudes distally from the probe body 15 at least in part and is provided with at least one guiding surface 32 that is skewed with respect to the longitudinal axis L. The guiding surface 32 is configured such that a lateral flow component is superimposed on the discharging gas stream 30, thereby enabling targeted shaping and widening of the plasma zone. This improves, in particular, ignition stability and effectiveness in lateral applications. The invention further relates to a system 10 comprising the plasma probe 11 and a supply unit 12 with a high-frequency generator 17 and a gas source 20 for plasma generation.

1. A plasma probe having a probe body extending from a proximal probe end toward a distal probe end and having at least one lumen configured for gas conduction, wherein at the distal probe end:
at least one gas outlet opening is formed, into which the lumen opens and from which a gas stream is dischargeable, and
at least one electrode having a flow-guiding body extending along a longitudinal axis (L) and protruding distally from the probe body at least in part, wherein the flow-guiding body has at least one guiding surface arranged in the gas stream, which guiding surface extends along an extension direction that is arranged skewed with respect to the longitudinal axis (L) and is configured to deflect the gas stream such that it discharges with an additional lateral flow component and forms a plasma zone widened in the lateral direction.

2. The plasma probe according to claim 1, wherein the flow-guiding body is configured to form the plasma zone such that the plasma zone has a variable diameter depending on a flow velocity of the gas stream.

3. The plasma probe according to claim 1, wherein the flow-guiding body has at least one flow groove, wherein the guiding surface is formed by a flank of the flow groove facing the gas stream.

4. The plasma probe according to claim 1, wherein the flow-guiding body has a first section arranged inside the probe body and a second section arranged outside the probe body.

5. The plasma probe according to claim 4, wherein the probe body is formed as a hose, wherein the first section of the flow-guiding body, together with a wall of the hose, forms the at least one gas outlet opening.

6. The plasma probe according to claim 1, wherein the at least one guiding surface extends in a spiral course around the longitudinal axis (L) of the flow-guiding body.

7. The plasma probe according to claim 1, wherein the flow-guiding body is made of electrically conductive material, wherein it preferably forms at least a part of the electrode.

8. The plasma probe according to claim 4, further comprising: a jet-forming device for dispensing an irrigation fluid, the jet-forming device comprising at least one irrigation opening through which jet-forming fluid is dispensable at the distal probe end.

9. The plasma probe according to claim 8, wherein the jet-forming device has a nozzle element which is arranged at least partially in a through-bore of the flow-guiding body.

10. The plasma probe according to claim 9, wherein the nozzle element is fluidically connected to a capillary tube extending from the proximal probe end to the distal probe end, in which a channel for jet-forming fluid is formed.

11. The plasma probe according to claim 9, wherein the nozzle element consists of an electrically conductive material and, together with the flow-guiding body, forms the electrode.

12. The plasma probe according to claim 1, wherein a plurality of depressions are formed on a surface of the flow-guiding body.

13. The plasma probe according to claim 1, wherein the extension direction of the guiding surface is arranged at a predetermined inclination angle relative to the longitudinal axis (L).

14. The plasma probe according to claim 1, wherein, in longitudinal section, the guiding surface has, at least in a central region of the guiding surface, a tangent which is arranged at a predetermined angle of attack relative to a radial direction of the flow-guiding body.

15. A system comprising a supply unit and the plasma probe according to claim 1, wherein the supply unit has:
a high frequency (HF) generator for supplying the at least one electrode with HF currents, and
a gas source configured to provide a gas for plasma generation at a flow velocity, wherein the flow velocity is variable, wherein
the plasma probe is connected to the supply unit such that the at least one lumen is fluidically connected to the gas source and a gas stream is dischargeable from the at least one gas outlet opening, and
that the electrode is electrically connected to the HF generator and configured to be supplied with HF currents.
